(19) United States
(12) Patent Application Publication
(10) Pub. No.: US 2005/0070519 A1
(43) Pub. Date: Mar. 31, 2005

(54) Cyclisation process for the preparation of c-2 beta-lactam compounds

(76) Inventors: Christopher Joseph Schofield, Oxford (GB); Kirsty Sarah Hewitson, Oxford (GB); Mark Christopher Sleeman, Oxford (GB)

Correspondence Address: CLARK & ELBING LLP, 101 FEDERAL STREET, BOSTON, MA, 02110, US

(21) Appl. No.: 10/499,230

(22) PCT Filed: Dec. 19, 2002
(86) PCT No.: PCT/GB02/05804

(30) Foreign Application Priority Data
Dec. 19, 2001 (GB) 0130351.0

(51) Int. Cl. A61K 31/397; C12P 37/00

(52) U.S. Cl. 514/210.02; 544/200; 435/43

(57) ABSTRACT

A process for the preparation of a substituted C-2 β-lactam comprises incubating a 2-substituted 3-aminocarboxylic acid with a β-lactam synthetase under conditions such that the 2-substituted 3-aminocarboxylic acid is cyclised to produce a substituted C-2 β-lactam. The process can be used to produce an antibiotic or β-lactamase inhibitor.

### FIELD OF THE INVENTION

The present invention relates to a process for the preparation of a substituted C-2 β-lactam which comprises incubating a 2-substituted 3-aminocarboxylic acid with a β-lactam synthetase or modified β-lactam synthetase either in vitro or within cells. This process may be used in the synthesis of β-lactam antibiotics.

### BACKGROUND OF THE INVENTION

The β-lactam antibiotics, which include penicillins, cephalosporins and carbapenems, contain a β-lactam ring as part of their chemical structure. This ring is vital to their antibiotic activity and is critical in preventing peptides from attaching to side chains during cell wall formation.

β-Lactam compounds are susceptible to degradation by β-lactamase enzymes which are produced by several clinically important microorganisms. β-lactamase inhibitors, such as clavulanic acid (1), have been used successfully in combination with β-lactam antibiotics in the treatment of infections caused by β-lactamase producing microorgansims.

A β-lactam synthetase is believed to act in the biosynthetic pathway to clavulanic acid, in the cyclisation of N²-(2-carboxyethyl)-(L)-arginine (CEA) (2) to deoxyguanidinoproclavaminic acid (DGPC) (3).

However, whilst clavulanic acid and many other β-lactam antibiotics and β-lactamase inhibitors based on the cephalosporins and penicillins may be produced from fermented materials already containing β-lactam nuclei, other sub-families of β-lactam antibiotics, such as the β-lactam antibiotics, such as the carbapenems (e.g. (5)), are currently produced by total synthesis. For example, a key intermediate in the preparation of a number of β-lactam antibiotics, such as carbapenems, trinems and oxapenems, is 3-(1-hydroxyethyl)-4-(acetyloxy)-2-azetidinone (4), which is produced by synthesis.

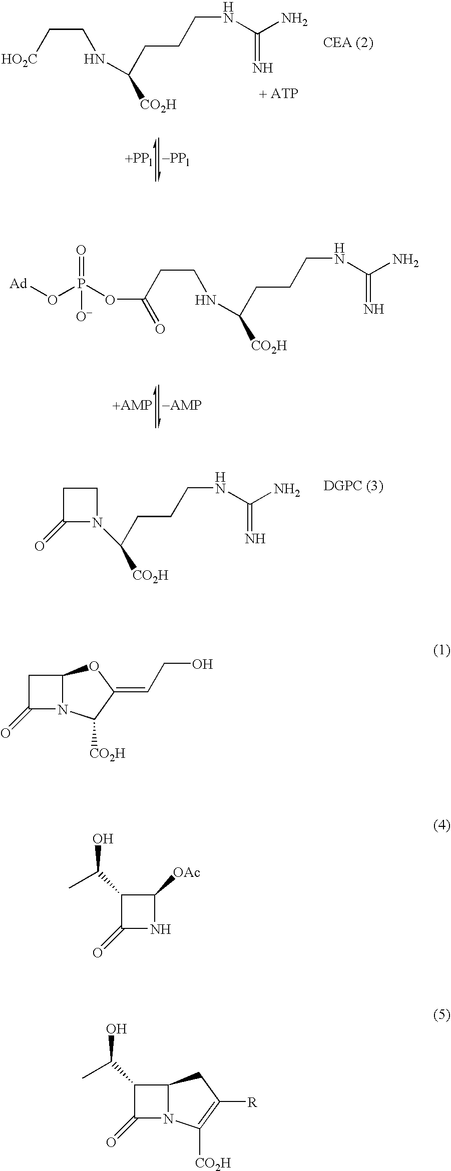

The production costs of antibiotics and β-lactamase inhibitors that must presently be prepared by total synthesis limits their use and precludes the development of new compounds.

### SUMMARY OF THE INVENTION

The present invention provides a process for the preparation of a substituted C-2 β-lactam comprising incubating a 2-substituted 3-aminocarboxylic acid with a β-lactam synthetase or modified β-lactam synthetase under conditions such that the 2-substituted 3-aminocarboxylic acid is cyclised to produce a substituted C-2-β-lactam.

In a preferred embodiment the process further comprises synthesising a carbapenem, trinem or oxapenem antibiotic from the substituted C-2 β-lactam so produced.

### DETAILED DESCRIPTION OF THE INVENTION

The present invention provides a process for the preparation of a substituted C-2 β-lactam which uses a β-lactam synthetase enzyme.

A substituted C-2 β-lactam is formed by cyclisation of a 2-substituted 3-aminocarboxylic acid by a β-lactam synthetase or a modified β-lactam synthetase. Preferably, the 2-substituted 3-aminocarboxylic acid comprises substituents which promote ring closure in the process of the invention. The 2-substituted 3-aminocarboxylic acid is preferably a molecule of the general formula (I): 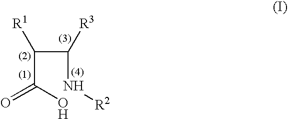
wherein R¹ is selected from C₁ to C₄ alkyl, C₁ to C₄ alkoxy, C₁ to C₄ alkylthio, C₁ to C₄ hydroxyalkyl, amino, amido, amidino, guanidino, benzyl, phenyl, R¹³CONH, C₆H₅CH₂CONH and C₆H₅OCH₂CONH. In a preferred embodiment, R¹ is selected from methyl, ethyl, hydroxyethyl (of R or S stereochemistry) and R¹³CONH; wherein R¹³ is selected from hydrogen, alkyl such as C₁ to C₁₀ alkyl, aryl, heteroaryl, aryl alkyl, aryl-C₂ to C₄ alkenyl or aryl-C₁ to C₄ alkyl such as an aryl methylene such as PhCH₂ or PhOCH₂; wherein aryl or heteroaryl are mono-ring, or have two fused rings one of which may be saturated, and which aryl and heteroaryl groups may be substituted by one or more C, to C₄ alkyl, halo, NR¹OR¹¹, SO₂R¹⁰R¹¹, CONR¹⁰R¹¹, C₁ to C₆ alkyl ester, CN, CH₂OH, O—C₁ to C₆ alkyl, CF₃ or nitro groups; wherein R¹⁰ and R¹¹, which may be the same or different, are hydrogen or C₁ to C₄ alkyl; wherein R³ may be selected from H, CH₃, CH₂CH₃, CO₂H, CONH₂, OAc, CO₂R¹⁴, CH₂OH and 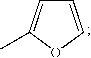
wherein R¹⁴ is selected from hydrogen, alkyl, preferably C₁ to C₁₀ alkyl, such as CH₃ or C₂H₅, and CH₂R¹⁵ wherein R¹⁵ is an aromatic group such as phenyl, 4-nitrophenyl or methoxyphenyl or an aryl, heteroaryl, aryl alkyl, aryl-C₂ to C₄ alkenyl or aryl-C₁ to C₄ alkyl; wherein aryl or heteroaryl are mono-ring, or have two fused rings one of which may be saturated, and which aryl and heteroaryl groups may be substituted by one or more C₁ to C₄ alkyl, halo, NR¹⁰R¹¹, SO₂R¹⁰R¹¹, CONR¹⁰R¹¹, C₁ to C₆ alkyl ester, CN, CH₂OH, O—C₁ to C₆ alkyl, CF₃ or nitro groups; wherein R¹⁰ and R¹¹, which may be the same or different, are hydrogen or C₁ to C₄ alkyl; and wherein R² is a readily functionalisable or cleavable group. Preferably, R² is selected from hydrogen and an aliphatic or aromatic substituent, for example CH₂CO₂H, SO₂R⁷, CO₂R⁷, CONHR⁷, COR⁷, C═CR⁸R⁹ wherein R⁷ is C₁ to C₆ alkyl; CH₂(CF₂)₀₋₄CF₃; aryl or heteroaryl, which aryl or heteroaryl are mono-ring, or have two fused rings one of which may be saturated, and which aryl and heteroaryl groups may be substituted by one or more C, to C₄ alkyl, halo, NR¹⁰R¹¹, SO₂R¹⁰R¹¹, CONR¹⁰R¹¹, C₁ to C₆ alkyl ester, CN, CH₂OH, O—C₁ to C₆ alkyl, CF₃ or nitro groups; aryl-C₁ to C₄ alkyl; aryl-C₁ to C₄-alkyl-NH; or aryl-C₂ to C₄ alkenyl; or such groups wherein aryl is substituted by one or more C₁ to C₄ alkyl or halo groups R⁸ and R⁹, which may be the same or different, are COR¹²; R¹⁰ and R¹¹, which may be the same or different, are hydrogen or C₁ to C₄ alkyl; and R¹² is hydrogen or an aliphatic, aromatic or heteroaromatic group. Preferably, R² is of the general formula —CR⁶—CO₂H, wherein R⁶ is hydrogen or an aliphatic group. Preferably R⁶ is of the general formula: 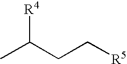
wherein R⁴ is selected from hydrogen and OH and R⁵ is selected from NH₂

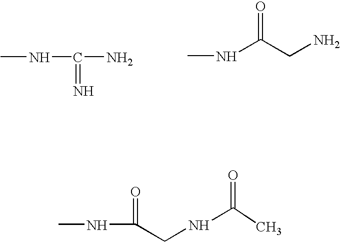

Accordingly, in a preferred aspect, the 2-substituted 3-aminocarboxylic acid is of the formula: 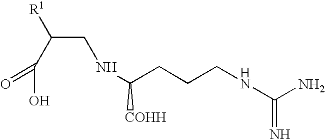
wherein R¹ is as defined above.

The present invention provides a process by which a β-lactam synthetase or modified β-lactam synthetase enzyme catalyses the cyclisation of a 2-substituted 3-aminocarboxylic acid, for example a molecule of formula (I), to a substituted C-2 β-lactam, for example a molecule of formula (II):

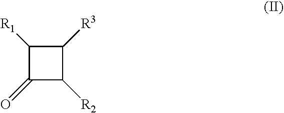

In a preferred aspect of the invention, the substituted C-2 β-lactam formed by cyclisation of the 2-substituted 3-aminocarboxylic acid is suitable as an intermediate in the production of a β-lactam antibiotic or β-lactamase inhibitor. For example, the substituted C-2 β-lactam may be a 2-azetidinone such as 3-(1-hydroxyethyl)-4-(acetyloxy)-2-azetidinone, or an intermediate suitable for use in the formation of this compound.

The 2-substituted 3-aminocarboxylic acid may be obtained from any suitable source, for example, by direct synthesis or by action of another enzyme either in vitro or in cells.

The present invention encompasses the use of 2-substituted 3-aminocarboxylic acid or salts thereof in the processes described herein. For example, the invention encompasses the salts of those compounds of formula (I) that have salt forming groups, such as an acidic or basic group. In the case of compounds comprising a basic amino group, the salts are formed with suitable inorganic or organic acids. Suitable inorganic acids are, for example, hydrochloric or sulphuric acid. Suitable organic acids include mono- di- and tricarboxylic acids such as acetic, trifluoroacetic, tartaric and citric acid, or sulphonic acids, for example methanesulphonic, trifluoromethanesulphonic orp-toluenesulphonic acid.

The present invention includes all possible isomers and mixtures thereof, including diastereomeric mixtures and racemic mixtures, resulting from the possible combinations of (R) and (S) stereochemistry.

For use in a process of the present invention, the 2-substituted 3-aminocarboxylic acid or a salt thereof is suitably dissolved, for example, in buffer before mixing with the enzyme. The concentration of precursor solution will depend upon the solubility of the precursor; usually the concentration of the precursor solution is in the range of from 5% w/v to 0.001% w/v.

A β-lactam synthetase for use in connection with the present invention is one has suitable β-lactam synthetase activity, that is, the ability to catalyse cyclisation of an 3-aminocarboxylic acid, in particular an 2-substituted 3-aminocarboxylic acid, to produce a β-lactam.

A β-lactam synthetase for use in the present invention may be a naturally occurring β-lactam synthetase, such as a β-lactam synthetase enzyme purified from a natural source, for example from Streptomyces clavuligerus. Streptomyces antibioticus, or other clavam or carbapenem producing Streptomyces species or from other microorganisms such as Erwinia carotovora or Serratia marcesceus.

Variations in the sequence of β-lactam synthetase may be present in β-lactam synthetase obtained from other microorganisms. A β-lactam synthetase for use in the present invention may therefore be a naturally occurring variant which is expressed by Streptomyces clavuligerus or another microorganism. Variant β-lactam synthetases include polypeptides which vary from the β-lactam synthetase of Streptomyces clavuligerus but are not necessarily naturally occurring β-lactam synthetases. Over the entire length of the amino acid sequence of the β-lactam synthetase of Streptomyces clavuligerus, a variant will preferably be at least 80% homologous to that sequence based on amino acid identity. More preferably, the polypeptide is at least 85% or 90% and more preferably at least 95%, 97% or 99% homologous to that amino acid sequence over the entire sequence. There may be at least 80%, for example at least 85%, 90% or 95%, amino acid identity over a stretch of 40 or more, for example 60, 100 or 120 or more, contiguous amino acids (“hard homology”).

Amino acid substitutions may be made to the β-lactam synthetase sequence of Streptomyces clavuligerus, for example from 1, 2 or 3 to 10, 20 or 30 substitutions. Conservative substitutions may be made, for example, according to the following table. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

One or more amino acid residues of the β-lactam synthetase amino acid sequence of Streptomyces clavuligerus may alternatively or additionally be deleted. From 1, 2 or 3 to 10, 20 or 30 residues may be deleted, or more. Enzymes for use in the processes of the invention also include fragments of the above-mentioned sequences retain β-lactam synthetase activity. Fragments may be at least from 10, 12, 15 or 20 to 60, 100 or 200 amino acids in length.

A β-lactam synthetase enzyme may be obtained from an organism such as Streptomyces clavuligerus by culturing the microorganism, harvesting and lysing the mycelium, and isolating the β-lactam synthetase enzyme. β-lactam synthetase from other Streptomyces clavuligerus strains or other bacterial species can be isolated following standard cloning techniques, for example, using the polynucleotide sequence of β-lactam synthetase from Streptomyces clavuligerus or a fragment thereof as a probe.

The β-lactam synthetase may be used in vitro or in cells. A cell-free enzyme extract may be produced, for example by sonication or other disruption of the microorganisms, optionally thereafter removing cell debris, leaving the β-lactam synthetase enzyme in solution. This solution may then be fractionated to isolate the β-lactam synthetase enzyme. The enzyme may be isolated and used in purified form, partially-purified form, as obtained in an impure state, as a filtrate from a disrupted cell preparation, as a crude cell homogenate, or in an immobilised form on a column.

For use in vitro, the enzyme may be in a substantially isolated form. It will be understood that the enzyme may be mixed with carriers or diluents which will not interfere with the intended purpose of the enzyme and still be regarded as substantially isolated.

The enzyme may also be in a substantially purified form, in which case it would generally comprise the enzyme in a preparation in which more than 90%, e.g. 95%, 98% or 99%, by weight of the enzyme in the preparation is an active β-lactam synthetase enzyme. Most suitably the enzyme is, for example, at least partially purified to remove other enzymes which might catalyse the destruction of the precursor, the enzyme, or the β-lactam nucleus once formed.

The enzyme may be recovered and purified from the disrupted cell preparation by well-known methods including ammonium sulphate or ethanol precipitation, acid extraction, anion or cation exchange chromatography, size exclusion chromatography, phosphocellulose chromatography, hydrophobic interaction chromatography, affinity chromatography, hydroxylapatite chromatography and lectin chromatography. Most preferably, high-performance liquid chromatography or a variant optimised for protein purification is employed for purification. Well-known techniques for refolding proteins may be employed to regenerate an active conformation when the polypeptide is denatured during isolation and/or purification.

The enzyme may be made synthetically or by recombinant means. The amino acid sequence of the enzyme may be modified to include non-naturally occurring amino acids or to increase the stability of the enzyme. Where the enzyme is produced by synthetic means, such amino acids may be introduced during production. The enzyme may also be modified following either synthetic or recombinant production. A modified β-lactam synthetase suitable for use in a process of the present invention will retain the activity of a β-lactam synthetase to cyclise a 3-aminocarboxylic acid, in particular the ability to cyclise a 2-substituted 3-aminocarboxylic acid.

The β-lactam synthetase enzyme may be modified for example by deletion or mutation such that the β-lactam synthetase activity is modified or increased. In particular, the activity of the enzyme for a substrate other than its natural substrate may be increased. For example, the modification may enhance the activity or specificity of the enzyme for a substrate which is substituted at the C-2 position. Preferably the modified β-lactam synthetase demonstrates improved activity in the cyclisation of a 2-substituted 3-aminocarboxylic acid to a substituted C-2 β-lactam compared to the corresponding enzyme which does not incorporate the selected modification. The β-lactam synthetase may be modified through amino acid substitution within the active site of the enzyme.

A β-lactam synthetase enzyme may be modified, for example by the addition of histidine residues to assist its identification or purification or by addition of a signal sequence to promote its secretion from a cell where the enzyme does not naturally contain such a sequence.

The β-lactamase enzyme may be modified by, for example, amino acid substitution, deletion or extension. A modified β-lactam synthetase enzyme may be a fragment of a naturally-occurring β-lactam synthetase enzyme which retains the β-lactam synthetase activity. The β-lactam synthetase enzyme may be extended at the N-terminus or C-terminus of the amino acid sequence of the enzyme. A carrier protein may be fused to the enzyme. A fusion protein incorporating an active β-lactam synthetase can thus be provided.

A number of side-chain modifications are known in the art and may be made to the side-chains of the enzyme. Such modifications include, for example, modifications of amino acids by reductive alkylation by reaction with an aldehyde followed by reduction with NaBH₄, amidination with methylacetimidate or acylation with acetic anhydride.

The amino acid sequence encoding the β-lactam synthetase enzyme may be introduced into a cell by in situ expression of the polypeptide from a recombinant expression vector. The expression vector optionally carries an inducible promoter to control the expression of the polypeptide.

An enzyme may be used in vitro, for example, bound to an immobile substrate such as an insoluble polymeric support. The enzyme may be immobilised through the addition of a binding sequence such as a His-tag or maltose binding site or by using a general immobiliser. The immobilised enzyme can then be used in the processes of the invention.

A compound or enzyme for use in the present invention may carry a revealing label. The revealing label may be any suitable label which allows the compound or enzyme to be detected. Suitable labels include radiostopes such as ¹²⁵I, ³³P or ³⁵S, enzyme labels, antibodies or linkers such as biotin. Such labels may be detected using techniques known per se.

A β-lactam synthetase or modified β-lactam synthetase in accordance with the invention is useful in the cyclisation of 2-substituted 3-aminocarboxylic acids. Such cyclisation may be carried out in vitro or in vivo. Such cyclisation may be used as part of the process for the production of a β-lactam antibiotic or β-lactamase inhibitor.

The process of the present invention is generally carried out in aqueous media, the reaction mixture suitably being maintained in the range of from pH 4 to 9, more suitably, for example, from 6.5 to 9.0, preferably about pH 8.5. The pH is suitably controlled, for example, using buffers, such as, for example, 3-(N-morpholino)propanesulphonic acid or tris(hydroxymethyl)aminomethane buffer at pH 9. Alternatively the pH may be controlled by the addition of a suitable acid or base. The temperature of the reaction should be that suitable for the enzyme employed and is generally in the range of from 15° C. to 60° C., preferably about 30-37° C. The reaction time depends on such factors as concentrations of reactant and cofactors, temperature and pH.

After the reaction is complete, the enzyme may be separated from the reaction mixture and the substituted C-2 β-lactam or a salt thereof, isolated by conventional methods, such as by chromatography, extraction into an organic solvent or by precipitation. The substituted C-2 β-lactam may be isolated in a form where the carboxyl and/or the amino group present is protected and, if desired, the protecting group(s) may be subsequently removed to generate the compound in a pure form.

Salts of the substituted C-2 β-lactam may be produced, for example, by treating the unsalified compound with the appropriate acid or base. The compounds, and salts thereof, produced by the above processes, may be recovered by conventional methods.

Substituted C-2 β-lactam possessing two chiral centres may be separated into diastereoisomeric pairs of enantiomers, if so desired, by, for example, fractional crystallisation from a suitable solvent, for example methanol or ethyl acetate or a mixture thereof. The pair of enantiomers or other pairs of enantiomers may be separated into individual stereoisomers by conventional means, for example by the use of an optically active salt as a resolving agent or by stereoselective removal of a protecting group using a suitable enzyme, for example an esterase such as subtilisin. In mixtures of diastereoisomers of the compounds the ratio of diastereoisomers may be changed by treatment with a non-nucleophilic base, for example, 1,5-diazabicyclo[4.3.0]non-5-ene.

Suitable optically active compounds which may be used as resolving agents are described in ‘Topics in Stereochemistry’, Vol.6, Wiley Interscience, 1971, Allinger, N. L. and Eliel, W. L., Eds.

Alternatively, any enantiomer of a substituted C-2 β-lactam may be obtained by stereospecific synthesis using optically pure substituted 4-amino carboxylic acid of known configuration.

In an alternative aspect of the invention, host cells may be provided which are transformed with polynucleotide encoding a β-lactam synthetase or modified β-lactam synthetase suitable for use in a process of the present invention. A polynucleotide encoding a β-lactam synthetase may be introduced into a replicable vector, for example a vector which is capable of providing for the expression of the coding sequence by the host cell. The vector may be for example, a plasmid, virus or phage vector provided with an origin of replication, optionally a promoter for the expression of the said polynucleotide and optionally a regulator of the promoter. The vectors may contain one or more selectable marker genes, for example a tetracycline resistance gene. Promoters and other expression regulation signals may be selected to be compatible with the host cell for which the expression vector is designed. Multiple copies of the same or different β-lactam synthetase gene in a single expression vector; or more than one expression vector each including a β-lactam synthetase gene which may be the same or different may be transformed into the host cell. The vector may then be introduced into a compatible host cell and the cell cultivated under conditions which bring about the expression of the polypeptide. Preferably, the host cells will be cells of bacterial or fungal origin such as E. coli, a Streptomyces SPP or Peincillium chrysogenum, particularly, for the production in vitro of the β-lactam synthetase for use in the invention.

The invention may therefore be operated using such an intact host cell expressing a β-lactam synthetase enzyme. The precursor 2-substituted 3-amino carboxylic acid, or salt thereof, is provided and contacted with the microorganism under conditions enabling conversion of the 2-substituted 3-amino carboxylic acid to the substituted C-2 β-lactam. Alternatively, the enzyme may be expressed in a host cell which is capable of producing a 2-substituted 3-aminocarboxylic acid such that the 2-substituted 3-aminocarboxylic acid is converted to a substituted C-2 β-lactam.

The host cell may be in the form of a growing culture, resting culture, washed mycelium, immobilised cells, or protoplasts.

In a further embodiment a cell-free system, derived from the recombinant organism, may be used to carry out the process of the invention. The cell-free extract may be prepared, and the enzyme purified, as hereinbefore described.

The substituted C-2 β-lactam obtained by the processes of the present invention may be active as an antibiotic, for example a carbapenem, trinem or oxapenem antibiotic or as a β-lactamase inhibitor. In another aspect, the substituted C-2 β-lactam produced by the processes of the invention is an intermediate in the synthesis pathway of an antibiotic, for example, a carbapenem, trinem or oxapenem antibiotic or of a β-lactamase inhibitor. The present invention therefore also provides a process wherein an antibiotic or β-lactamase inhibitor is synthesised from the C-2 β-lactam. Preferably, a carbapenem, trinem or oxapenem antibiotic is synthesised from the C-2 β-lactam. Synthesis may be carried out by routine methods known to the person skilled in the art. The following examples further illustrate the present invention:

### EXAMPLES

2-Methyl CEA was prepared by substituting methyl acrylic acid for acrylic acid in the reported method for preparation of CEA (Baldwin, J. E.; Lloyd, M. D.; Whason, B.; Schofield, C. J.; Elson, S. W.; Baggaley, K. H.; Nicholson, N. H. J. Chem. Soc., Chem. Commun. 1993, 500-502).

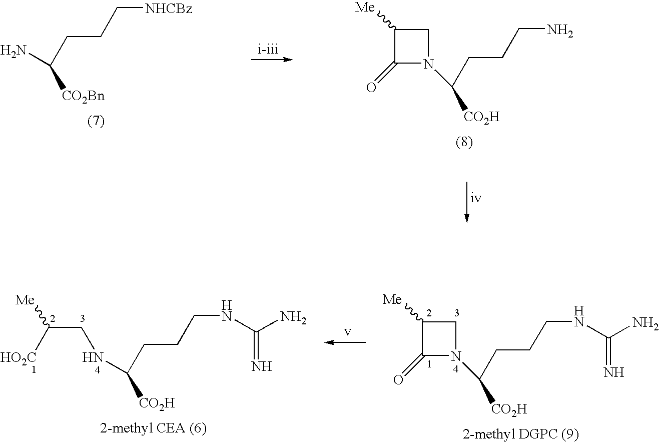

Michael reaction of protected ornithine (7) with 2-methyl acrylic acid, followed by ring closure and deprotection gave epimeric monocyclic β-lactam (8). Guanylation followed by hydrolysis gave the desired analogue (6) as a mixture of epimers.

The reagents for each of the steps shown above were as follows: (i) CH₂═CMeCO₂H, MeCN, 60° C., 60%; (ii) MeSO₂Cl, NaHCO₃(aq.), MeCN, 60° C.; (iii) 10% Pd/C/H₂, EtOH:H₂O (2:1); (iv) 1-amidino-3,5-dimethylpyrazole-HNO₃, dimethylformamide-H₂O, pH 8-9, 45% plus 40% recovered (8); (v) 1M HCl 1 hr.

The bls gene has previously been expressed at relatively low levels using a pET24a(+) construct and purified by an involved two column protocol giving low overall yields (McNaughton, H. J.; Thirkettle, J. E.; Zhang, Z. H.; Schofield, C. J.; Jensen, S. E.; Barton, B.; Greaves, P. J. Chem. Soc., Chem. Commun. 1998, 2325-2326; Bachmann, B. O.; Townsend, C. A. Biochemistry 2000, 39, 11187-11193). To facilitate purification a polyhistidine tagged form of BLS (β-lactam synthetase) was produced by cloning bis into the pET28a(+) vector (from Novagen). BLS produced using this vector was isolated to >95% purity (by SDS PAGE analysis) using a single affinity purification step with a Nickel His bind™ column.

The bls gene in the vector pET28a(+) and a plasmid bearing the chaperonin GroELS¹³ were simultaneously transformed into E. coli BL21(DE3) for co-expression. Cells were grown in 2TY containing 30 μg/ml kanamycin at 37° C. When the absorbance at 600 nm reached ˜1.0, IPTG was added to a final concentration of 1.0 mM and the cells harvested 16 hours later by centrifugation. Expression of BLS was observed as 15% of the total cell protein in the soluble fraction (by SDS-PAGE analysis). Cells were resuspended in binding buffer (20 mM Tris-HCl pH 7.9, 500 mM NaCl, 5 mM imidazole) and lysed by sonication. The lysate was centrifuged at 40000 g, and the supernatant loaded onto a pre-equilibrated 10 ml HisBind™ column. The column was washed with 100 ml binding buffer followed by 60 ml wash buffer (binding buffer with 60 mM imidazole). Protein was eluted with elute buffer (binding buffer with 200 mM imidazole). The protein was desalted through a PD-10 column (Pharmacia) and concentrated to 10 mg/ml. BLS was aliquoted into 50 μl portions and stored at −80° C.

The 2-methylated substrate analogue (6) was initially assayed as a substrate for BLS using a fluorescence detection pre-column derivatised HPLC assay based on the work of Kai et al (Kai, M.; Miyazaki, T.; Yamaguchi, M.; Ohkura, Y. J. Chromatogr. 1983, 425-436). Assay mixtures contained: 150 mM Tris-HCl, pH 9, 5 mM ATP, 10 mM MgCl₂, 2 mM 2-methyl CEA, ca. 74 μg enzyme.

Reactions were incubated at 37° C. for 15 minutes and then derivatised. Derivatisation conditions: To 50 μl assay mixture were added 25 μl benzoin (40 mM) in 2-methoxyethanol, 25 μl sodium sulphite (0.2 M)/β-mercaptoethanol (0.1 M), 50 μl KOH (2 M). The mixture was cooled on ice for 2 minutes, heated to 100° C. for 5 minutes and then cooled on ice for a further 2 minutes. 50 μl of Tris-HCl pH 9.2 was then added and the sample centrifuged for 30 seconds at 11000 g. 150 μl of the above sample was injected on to a reverse phase Hypersil phenyl column (250 mm×4.6 mm) and the elution of compounds compared to that of standards. A linear gradient of 15% (v/v) Tris-HCl (0.5 M) pH 8.5, 50-80% (v/v) MeOH over 16 minutes remaining at 80% (v/v) MeOH for 6 minutes, reverting to 50% (v/v) MeOH over 1 minute and re-equilibrating under these conditions for a further 15 minutes was run at 0.7 ml/min. Fluorescence was measured at 425 nm against 325 nm excitation.

Under the standard assay conditions ca. 50% of the 2-methyl CEA (6) was converted to 2-methyl DGPC (9), which was observed as a single HPLC peak coincident with that produced when using authentic (9). The HPLC assays were also carried out using wildtype BLS to ensure the polyhistidine tag was not affecting the conformation of the protein such that it accepted the alternative substrate. A similar result was obtained.

Incomplete substrate conversion may have in part resulted from partial epimerisation at the α-centre derived from arginine during the ring closure process in the synthesis, or from preferential processing of one of the 2-methyl epimers of (6). To investigate this a larger scale incubation was used to allow analysis by ¹H NMR (500 MHz). Peaks were observed at δ 4.2-4.5 consistent with conversion of ATP to AMP (FIG. 3).

The appearance of peaks corresponding to 2-methyl DGPC product (9) was also observed, including δ 4.05 (epimer 1, dd, J 10.0, 4.5 Hz, CHCO₂H), δ 4.0 (epimer 2, dd, J9.5, 5.5 Hz, CHCO₂H) and δ 1.5-2.0 (CH₂CH₂CH₂N & CH₂CH₂CH₂N)(FIG. 4).

The β-lactam product was isolated by reverse phase HPLC (C18 250×10 mm). Samples were eluted isocratically with a mobile phase of 10% (v/v) MeOH at a flow rate of 4 ml/minute. ESI MS analysis of the product was consistent with the production of 2-methyl DGPC (9) (m/z 243 [M−H]⁺). ¹H NMR (500 MHz), including 2D ¹H COSY analysis of the product was consistent with the formation of both 2 -methyl epimers of the product. There appeared to be a slight excess (<5%) of one epimer in the starting material (6) (by ¹H NMR analysis), that was apparently also present in the enzymic product (9), indicating that both epimers are approximately equally efficient substrates for BLS.

1. A process for the preparation of a substituted C-2 β-lactam comprising incubating a 2-substituted 3-aminocarboxylic acid with a β-lactam synthetase under conditions such that the 2-substituted 3-aminocarboxylic acid is cyclised to produce a substituted C-2 β-lactam.

2. A process according to claim 1 wherein said 2-substituted 3-aminocarboxylic acid is of the general formula 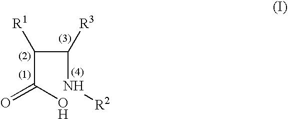
wherein R¹ is selected from C₁ to C₄ alkyl, C₁ to C₄ alkoxy, Ca to C₄ alkylthio, C₁ to C₄ hydroxyalkyl, amino, amidino, guanidino, benzyl, phenyl, R¹³CONH, C₆H₅CH₂CONH and C₆H₅OCH₂CONH; wherein R¹³ is selected from hydrogen, alkyl, aryl; heteroaryl, aryl alkyl, aryl-C₂ to C₄ alkenyl or aryl-C₁ to C₄ alkyl; wherein aryl or heteroaryl are mono-ring, or have two fused rings one of which may be saturated, and which aryl and heteroaryl groups may be substituted by one or more C₁ to C₄ alkyl, halo, NR¹⁰R¹¹, SO₂R¹⁰ R¹¹, CONR¹⁰R¹¹, C₁ to C₆ alkyl ester, CN, CH₂OH, O—C₁ to C₆ alkyl CF or nitro groups: wherein R¹⁰ and R¹¹, which may be the same or different, are hydrogen or C₁ to C₄ alkyl; wherein R² is selected from hydrogen and an aliphatic or aromatic substituent; and wherein R³ is selected from hydrogen, CH₃, CH₂CH₃, CO₂H, CONH₂, OAc, CO₂R¹⁴, CH₂OH and 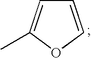
wherein R¹⁴ is selected from hydrogen, alkyl and CH₂R¹⁵ wherein R¹⁵ is an aromatic group.

3. A process according to claim 2 wherein R¹ is selected from methyl and hydroxyethyl.

4. A process according to claim 2 wherein R² is of the general formula —CR⁶—CO₂H, wherein R⁶ is hydrogen or an aliphatic group.

5. A process according to claim 4 wherein R⁶ is of the general formula 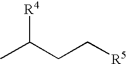
wherein R⁴ is selected from hydrogen or —OH and R⁵ is selected from

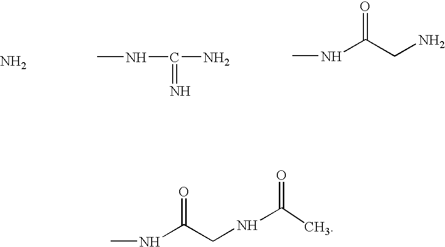

6. A process according to claim 1 wherein said β-lactam synthetase is a naturally occurring β-lactam synthetase.

7. A process according to claim 6 wherein said β-lactam synthetase is obtained from Streptomyces clavuligerus.

8. A process according to claim 1 wherein said β-lactam synthetase is a modified β-lactam-synthetase.

9. A process according to claim 8 wherein said modification increases the β-lactam synthetase activity for the 2-substituted 4-aminocarboxylic acid.

10. A process according to claim 1 which is carried out in vitro.

11. A process according to claim 1 wherein said β-lactam synthetase is provided by a host cell which expresses the β-lactam synthetase.

12. A process according to claim 1 wherein said substituted C-2 β-lactam is an antibiotic or β-lactamase inhibitor.

13. A process according to claim 1 further comprising synthesising an antibiotic or β-lactamase inhibitor from said substituted C-2 β-lactam.
